(19) United States
(12) Patent Application Publication
(10) Pub. No.: US 2023/0040492 A1
(43) Pub. Date: Feb. 9, 2023

(54) DETECTION DEVICE, DETECTION SYSTEM, DETECTION METHOD, AND PROGRAM RECORDING MEDIUM

(71) Applicant: NEC Corporation, Minato-ku, Tokyo (JP)

(72) Inventors: Chenhui HUANG, Tokyo (JP); Kenichiro FUKUSH, Tokyo (JP)

(73) Assignee: NEC Corporation, Minato-ku, Tokyo (JP)

(21) Appl. No.: 17/790,357

(22) PCT Filed: Jan. 8, 2020
(86) PCT No.: PCT/JP2020/000293

§ 371 Date: Jun. 30, 2022

(51) Int. Cl. A61B 5/11; A61B 5/00

(52) U.S. Cl. CPC A61B 5/112; A61B 5/7282; A61B 5/6829; A61B 5/1114; A61B 2562/0219

(57) ABSTRACT

A detection device that includes an extraction unit that extracts waveform data based on a gait of a walker using sensor data acquired from a sensor installed on a foot of the walker and a detection unit that detects a gait event from the waveform data extracted by the extraction unit.

### TECHNICAL FIELD

The present invention relates to a detection device or the like that detects a gait event of a walker.

### BACKGROUND ART

In response to growing interest in healthcare that manages physical condition, a service that measures a gait including a gait feature of a walker and provides information to users according to their gait has attracted attention. In order to measure the gait of the walker, it is necessary to detect gait events such as an event in which the foot landing on the ground or an event in which the foot off the ground from the data regarding walking.

PTL 1 discloses a method of acquiring data of plantar pressure from a pressure sensor provided in an insole installed in a shoe, analyzing the acquired data, and acquiring parameters related to the gait during walking or resting.

PTL 2 discloses a gait evaluation system that calculates a gait evaluation value of a subject by using acceleration data in three axial directions acquired by an acceleration sensor attached to an ankle of the subject.

### CITATION LIST

### Patent Literature

[PTL 1] WO 2018/164157

[PTL 2] JP 2019-150329 A

### SUMMARY OF INVENTION

### Technical Problem

According to the method of PTL 1, in a case where the pressure sensor is provided in the insole of the shoe, a gait state of the walker can be analyzed using data acquired by the pressure sensor. However, the method of PTL 1 cannot be applied to a case where the pressure sensor is not provided in the shoe insole.

According to the method of PTL 2, in a case where the acceleration sensor is attached to the ankle, the gait state of the walker can be analyzed using the data acquired by the acceleration sensor. However, the method of PTL 2 cannot be applied to a case where the acceleration sensor is not attached to the ankle.

An object of the present invention is to provide a detection device or the like capable of detecting a gait event in a gait of a walker based on data acquired by a sensor installed on a foot of the walker.

### Solution to Problem

A detection device according to one aspect of the present invention includes an extraction unit that extracts waveform data based on a gait of a walker using sensor data acquired from a sensor installed on a foot of the walker and a detection unit that detects a gait event from the waveform data extracted by the extraction unit.

In one aspect of a detection method of the present invention, a computer extracts waveform data based on a gait of a walker using sensor data acquired from a sensor installed on a foot of the walker and detects a gait event from the extracted waveform data.

A program according to one aspect of the present invention causes a computer to execute a process for extracting waveform data based on a gait of a walker using sensor data acquired from a sensor installed on a foot of the walker and a process for detecting a gait event from the extracted waveform data.

### Advantageous Effects of Invention

According to the present invention, it is possible to provide a detection device or the like capable of detecting a gait event in a gait of a walker based on data acquired by a sensor installed on a foot of the walker.

### BRIEF DESCRIPTION OF DRAWINGS

FIG. 1 is a diagram illustrating an example of a configuration of a detection system according to a first example embodiment.

FIG. 2 is a conceptual diagram illustrating an example in which a data acquisition device of the detection system according to the first example embodiment is installed in footwear.

FIG. 3 is a conceptual diagram for illustrating a relationship between a local coordinate system and a world coordinate system set for the data acquisition device of the detection system according to the first example embodiment.

FIG. 4 is a conceptual diagram for illustrating a plantar angle calculated by the detection device of the detection system according to the first example embodiment.

FIG. 5 is a conceptual diagram for illustrating a gait event.

FIG. 6 is a block diagram illustrating an example of a configuration of a data acquisition device of the detection system according to the first example embodiment.

FIG. 7 is a block diagram illustrating an example of a configuration of the detection device of the detection system according to the first example embodiment.

FIG. 8 is a conceptual diagram for illustrating a position of a marker attached to the periphery of a shoe for measuring a gait of a subject.

FIG. 9 is a conceptual diagram for illustrating arrangement of a camera for measuring the gait of the subject.

FIG. 10 is a graph illustrating an example of a gait cycle dependency of a height in a gravity direction (a height in a Z-direction) measured by the detection system according to the first example embodiment.

FIG. 11 is a graph for illustrating that the detection system according to the first example embodiment detects a timing of a toe off from gait waveform data of an acceleration in a traveling direction (Y-direction acceleration).

FIG. 12 is a graph for verifying a correlation between a timing when the toe off is detected by the detection system according to the first example embodiment and a timing when the toe off is detected by motion capture.

FIG. 13 is a graph for illustrating that the detection system according to the first example embodiment detects a timing of heel contact from the gait waveform data of acceleration in the traveling direction (the Y-direction acceleration) and the gait waveform data of an acceleration in a gravity direction (the height in the Z-direction).

FIG. 14 is a graph for verifying a correlation between a timing when the heel contact is detected by the detection system according to the first example embodiment and a timing when the heel contact is detected by motion capture.

FIG. 15 is a flowchart for illustrating an example of an operation of an extraction unit of the detection device of the detection system according to the first example embodiment.

FIG. 16 is a flowchart for illustrating an example of an operation of a detection unit of the detection device of the detection system according to the first example embodiment.

FIG. 17 is a flowchart for illustrating an example of an operation in which the detection unit of the detection device of the detection system according to the first example embodiment detects a timing of the start of swing phase.

FIG. 18 is a flowchart for illustrating an example of an operation in which the detection unit of the detection device of the detection system according to the first example embodiment detects a timing of the start of stance phase.

FIG. 19 is a block diagram illustrating an example of a configuration of a detection device according to a second example embodiment.

FIG. 20 is a block diagram for illustrating an example of a hardware configuration for achieving the detection device of the detection system according to each example embodiment.

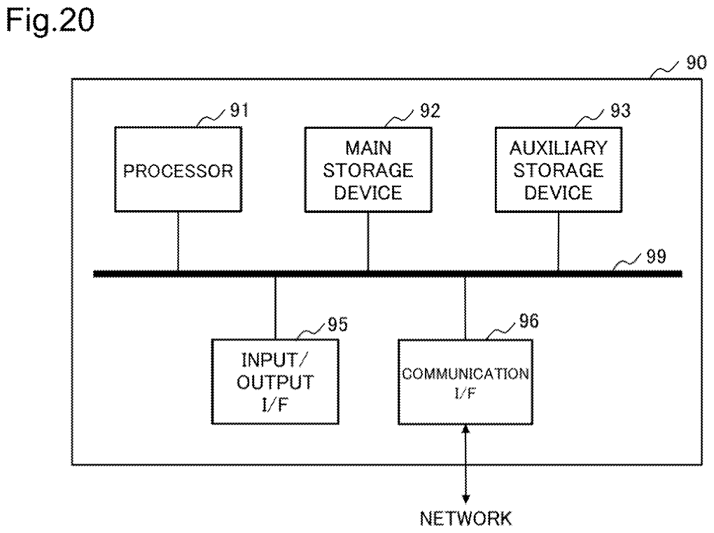

### EXAMPLE EMBODIMENT

Hereinafter, example embodiments of the present invention will be described with reference to the drawings. However, the example embodiments described below have technically preferable limitations for carrying out the present invention, but the scope of the invention is not limited to the following. In all the drawings used in the following description of the example embodiment, the same reference numerals are given to the same parts unless there is some particular reason not to. Further, in the following example embodiments, repeated description of similar configurations and operations may be omitted.

### First Example Embodiment

First, a detection system according to a first example embodiment will be described with reference to the drawings. The detection system of the present example embodiment detects the gait event of a walker using sensor data acquired by a sensor installed on a foot. Walk events include an event in which the foot lands the ground, an event in which the foot off the ground, and the like. Details of the gait event will be described later.

(Configuration)

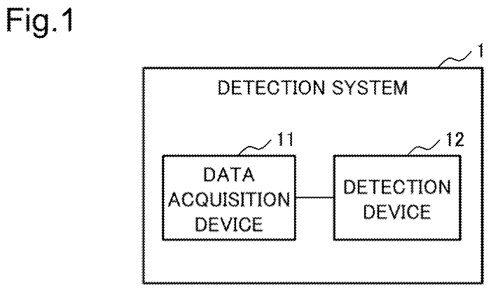

FIG. 1 is a block diagram illustrating an example of a configuration of a detection system 1 in the present example embodiment. As illustrated in FIG. 1, the detection system 1 includes a data acquisition device 11 and a detection device 12. The data acquisition device 11 and the detection device 12 may be connected by wire or wirelessly. In addition, the data acquisition device 11 and the detection device 12 may be configured by a single device. In addition, the data acquisition device 11 may be omitted from the configuration of the detection system 1 and the detection system 1 may be configured by the detection device 12 alone.

The data acquisition device 11 is installed on a foot. The data acquisition device 11 measures a spatial acceleration and a spatial angular velocity. The data acquisition device 11 generates sensor data based on the measured spatial acceleration and spatial angular velocity. The data acquisition device 11 transmits the generated sensor data to the detection device 12.

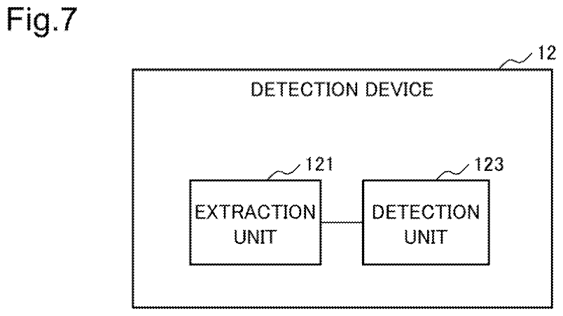

As illustrated in FIG. 7, the detection device 12 includes an extraction unit 121 and a detection unit 123. The extraction unit 121 extracts waveform data based on a gait of a walker using sensor data acquired from a sensor installed on a foot of the walker. The detection unit 123 detects a gait event from the waveform data extracted by the extraction unit 121.

The detection system 1 of the present example embodiment can be applied to detection of a gait event. Next, an example of a configuration of the detection system 1 that enables detection of the gait event will be described in detail.

The data acquisition device 11 includes at least an acceleration sensor and an angular velocity sensor. For example, the data acquisition device 11 is installed in an insole that is inserted into footwear. For example, the data acquisition device 11 is installed at a position below the arch of the foot. The data acquisition device 11 converts physical quantities such as acceleration and angular velocity acquired by the acceleration sensor and the angular velocity sensor into digital data (also referred to as sensor data). The data acquisition device 11 transmits the converted sensor data to the detection device 12. The data acquisition device 11 may be installed in any of the middle, the inside, and the surface of the footwear as long as waveform data similar to that at the position on the lower side of the arch of the foot can be obtained.

The data acquisition device 11 is achieved by, for example, an inertial measurement device including an acceleration sensor and an angular velocity sensor. An example of the inertial measurement device includes an inertial measurement unit (IMU). The IMU includes a three-axis acceleration sensor and a three-axis angular velocity sensor. Examples of the inertial measurement device include a vertical gyro (VG) and an attitude heading (AHRS). Further, as an example of the inertial measurement unit, there is a Global Positioning System/Inertial Navigation System (GPS/INS).

Sensor data such as acceleration and angular velocity acquired by the data acquisition device 11 is also referred to as gait parameters. In addition, a speed, an angle, a sensor height, and the like calculated by integrating the acceleration and the angular velocity are also included among the gait parameters. In the present example embodiment, a lateral direction of a walker is an X-direction (the right side is positive), a traveling direction of the walker is a Y-direction (the front side is positive), and a gravity direction is a Z-direction (upper side is positive). In the present example embodiment, rotation around the X-axis is defined as a roll, rotation around the Y-axis is defined as a pitch, and rotation around the Z-axis is defined as a yaw.

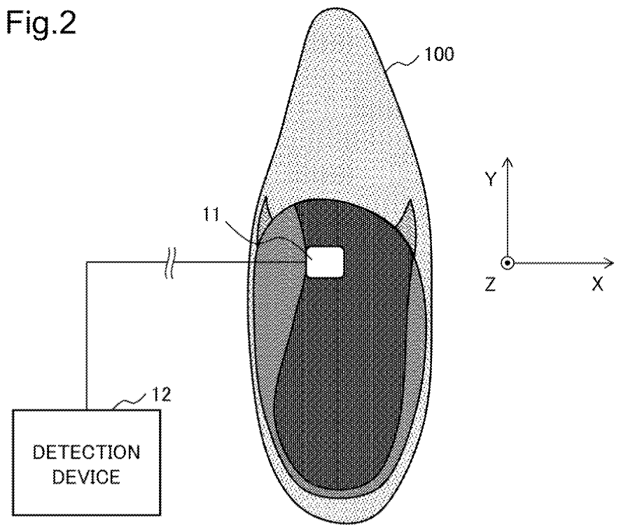

FIG. 2 is a conceptual diagram illustrating an example in which the data acquisition device 11 is installed in a shoe 100. In the example of FIG. 2, the data acquisition device 11 is installed at a position corresponding to the back side of the arch of the foot. For example, the data acquisition device 11 is installed in the insole inserted into the shoe 100. The data acquisition device 11 may be installed at a position other than the back side of the arch of the foot as long as waveform data similar to that at the back side of the arch of the foot can be obtained.

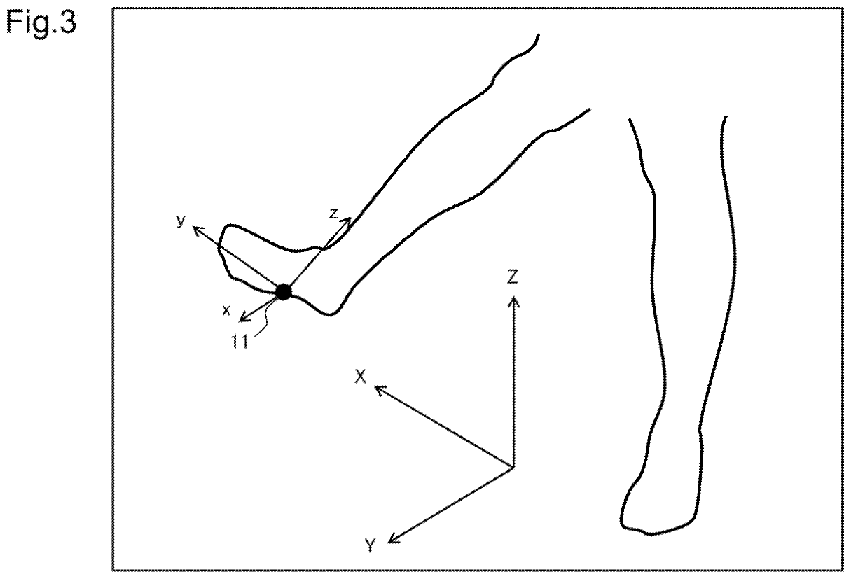

FIG. 3 is a conceptual diagram for illustrating a local coordinate system (x-axis, y-axis, and z-axis) set in the data acquisition device 11 and a world coordinate system (X-axis, Y-axis, and Z-axis) set with respect to the ground in a case where the data acquisition device 11 is installed on the back side of the arch of the foot. In the world coordinate system (X-axis, Y-axis, and Z-axis), in a state where a walker is standing upright, a lateral direction of the walker is set as an X-axis direction (the rightward direction is positive), a direction in front of the walker (the traveling direction) is set as a Y-axis direction (the forward direction is positive), and a gravity direction is set as a Z-axis direction (the vertically upward direction is positive). In a state where the walker is standing upright, the local coordinate system (x-axis, y-axis, and z-axis) and the world coordinate system (X-axis, Y-axis, and Z-axis) coincide with each other. In response to the walking of the walker, the spatial posture of the data acquisition device 11 changes. That is, in response to the walking of the walker, a difference occurs between the local coordinate system (x-axis, y-axis, and z-axis) and the world coordinate system (X-axis, Y-axis, and Z-axis). Therefore, the detection device 12 converts the sensor data acquired by the data acquisition device 11 from the local coordinate system (x-axis, y-axis, and z-axis) of the data acquisition device 11 into the world coordinate system (X-axis, Y-axis, and Z-axis).

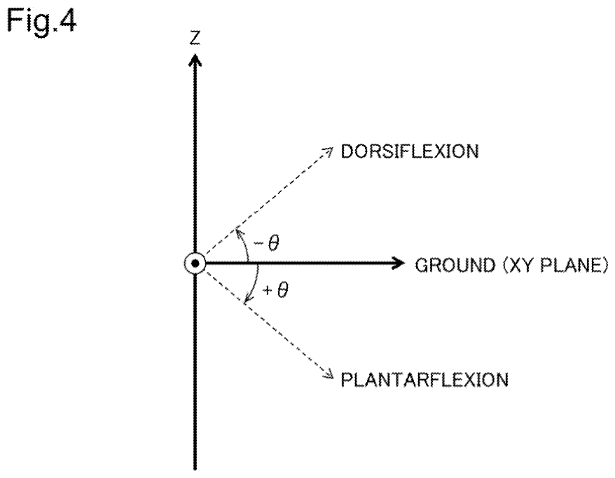

FIG. 4 is a conceptual diagram for illustrating a plantar angle calculated by the detection device 12. The plantar angle is the angle of the plantar surface relative to the ground (XY plane). The plantar angle is defined as negative when the toe is directed upward (dorsiflexion) and positive when the toe is directed downward (plantarflexion).

For example, the detection device 12 calculates the plantar angle using the magnitude of the acceleration in the axial direction of each of the X-axis and the Y-axis. Furthermore, for example, the detection device 12 can calculate the plantar angle about each of the X-axis, the Y-axis, and the Z-axis by integrating the values of the angular velocity having each of the X-axis, the Y-axis, and the Z-axis as the central axis. The acceleration data and the angular velocity data include high-frequency and low-frequency noises that change in various directions. Therefore, by applying a low-pass filter and a high-pass filter to the acceleration data and the angular velocity data to remove a high-frequency component and a low-frequency component, it is possible to improve accuracy of sensor data from a foot portion prone to noise. In addition, by applying a complementary filter to each of the acceleration data and the angular velocity data and taking a weighted average, the accuracy of the sensor data can be improved.

The detection device 12 acquires sensor data in the local coordinate system from the data acquisition device 11. The detection device 12 converts the acquired sensor data in the local coordinate system into the world coordinate system to generate time-series data. The detection device 12 extracts waveform data for one gait cycle (hereinafter, also referred to as gait waveform data) from the generated time-series data. The detection device 12 detects the gait event from the extracted gait waveform data. For example, the detection device 12 detects the gait event such as a timing when the toe leaves from the ground or a timing when the heel strikes the ground from the gait waveform data. The gait event detected by the detection device 12 is used as a reference when the gait of the walker is measured.

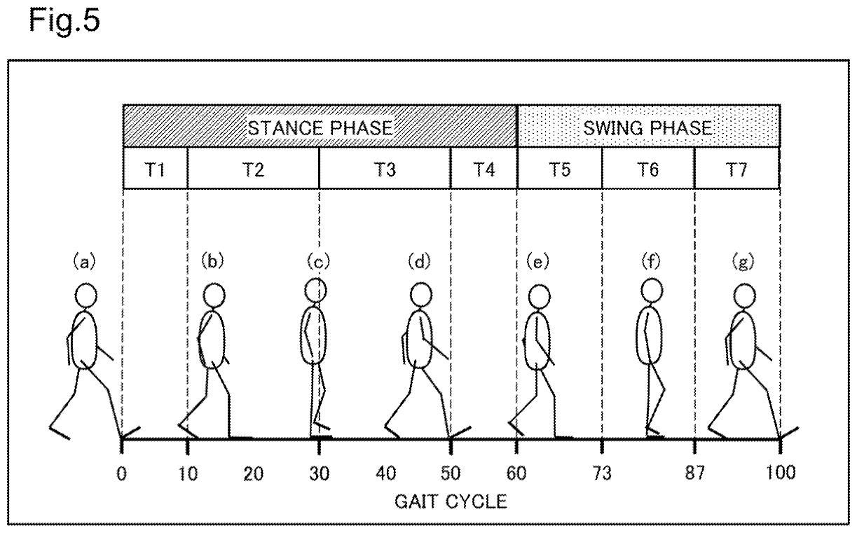

FIG. 5 is a conceptual diagram for illustrating a gait event. FIG. 5 illustrates one gait cycle of the right foot. The horizontal axis of FIG. 5 is a gait cycle normalized with one gait cycle of the right foot as 100%, with a time point at which the heel of the right foot strikes the ground as a starting point and a time point at which the heel of the right foot next strikes on the ground as an ending point. In general, one gait cycle of one foot is roughly divided into a stance phase in which at least a part of the back side of the foot is in contact with the ground and a swing phase in which the back side of the foot is off the ground. The stance phase is subdivided into an initial stance period T1, a mid-stance period T2, a terminal stance period T3, and a pre-swing period T4. The swing phase is further subdivided into an initial swing period T5, a mid-swing period T6, and a terminal swing period T7.

In FIG. 5, (a) represents a situation in which the heel of the right foot strikes the ground (heel contact). (a) is a starting point of one gait cycle illustrated in FIG. 5. (b) represents a situation in which the toe of the left foot leaves the ground in a state in which the entire sole of the right foot is on the ground (opposite foot too off). (c) represents a situation in which the heel of the right foot is lifted with the entire sole of the right foot on the ground (heel lift). (d) is a situation where the heel of the left foot is in contact with the ground (opposite foot heel contact). (e) represents a situation in which the toe of the right foot leaves the ground in a state in which the entire sole of the left foot is on the ground (too off). (f) represents a situation in which the left foot and the right foot cross each other in a state where the entire sole of the left foot is on the ground (foot crossing). (g) represents a situation in which the heel of the right foot strikes the ground (heel contact). (g) is the end point of one gait cycle illustrated in FIG. 5, and is the start point of the next gait cycle.

The detection device 12 detects time t_{d} when the plantar angle is minimum (dorsiflexion peak) and time t_{b} when the plantar angle is maximum (plantarflexion peak) after the dorsiflexion peak from the time-series data of the plantar angle. Further, the detection device 12 detects time t_{d+1} of the next dorsiflexion peak of the plantarflexion peak and time t_{b+1} of the next plantarflexion peak of the dorsiflexion peak. The detection device 12 cuts out waveform data for one gait cycle (gait waveform data) with the tₘ at the midpoint between the time t_{d} and the time t_{b} as a start point and time tₘ₊₁ at the midpoint between the time t_{d+1} and the time t_{b+1} as an end point. In the gait waveform data cut out by the detection device 12, the maximum (plantarflexion peak) appears at the time t_{b}, and the minimum (dorsiflexion peak) appears at time t_{d+1}.

[Data Acquisition Device]

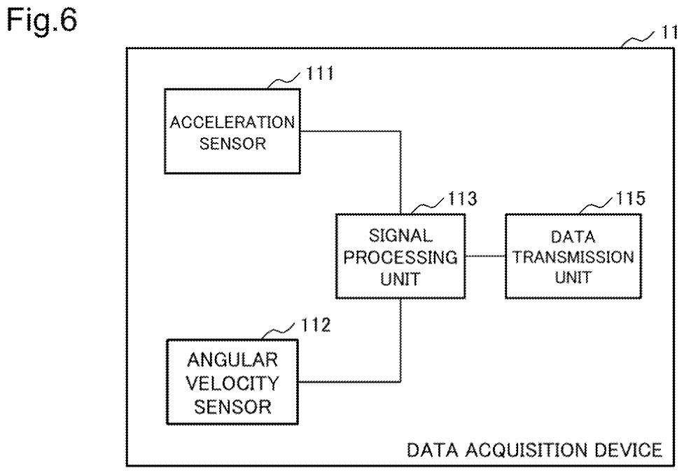

Next, details of the data acquisition device 11 included in the detection system 1 will be described with reference to the drawings. FIG. 6 is a block diagram illustrating an example of a configuration of the data acquisition device 11. The data acquisition device 11 includes an acceleration sensor 111, an angular velocity sensor 112, a signal processing unit 113, and a data transmission unit 115.

The acceleration sensor 111 is a sensor that measures accelerations in three axial directions. The acceleration sensor 111 outputs the measured acceleration to the signal processing unit 113.

The angular velocity sensor 112 is a sensor that measures angular velocities in three axial directions. The angular velocity sensor 112 outputs the measured angular velocity to the signal processing unit 113.

The signal processing unit 113 acquires acceleration and angular velocity from the acceleration sensor 111 and the angular velocity sensor 112, respectively. The signal processing unit 113 converts the acquired acceleration and angular velocity into digital data, and outputs the converted digital data (also referred to as sensor data) to the data transmission unit 115. The sensor data includes at least acceleration data (including acceleration vectors in three axial directions) obtained by converting analog data of acceleration into digital data and angular velocity data (including angular velocity vectors in three axial directions) obtained by converting analog data of angular velocity of into digital data. Acquisition times of the acceleration data and the angular velocity data are associated with the acceleration data and the angular velocity data. The signal processing unit 113 may be configured to output sensor data obtained by adding correction such as a mounting error, temperature correction, and linearity correction to the acquired acceleration data and angular velocity data.

The data transmission unit 115 acquires sensor data from the signal processing unit 113. The data transmission unit 115 transmits the acquired sensor data to the detection device 12. The data transmission unit 115 may transmit the sensor data to the detection device 12 via a wire such as a cable, or may transmit the sensor data to the detection device 12 via wireless communication. For example, the data transmission unit 115 can be configured to transmit sensor data to the detection device 12 via a wireless communication function (not illustrated) conforming to a standard such as Bluetooth (registered trademark) or WiFi (registered trademark). The communication function of the data transmission unit 115 may conform to a standard other than Bluetooth (registered trademark) or WiFi (registered trademark).

[Detection Device]

Next, details of the detection device 12 included in the detection system 1 will be described with reference to the drawings. FIG. 7 is a block diagram illustrating an example of a configuration of the detection device 12. The detection device 12 includes the extraction unit 121 and the detection unit 123.

The extraction unit 121 acquires sensor data from the data acquisition device 11 (sensor) installed on the footwear. The extraction unit 121 extracts, using the sensor data, gait waveform data associated with a gait of a walker wearing footwear on which the data acquisition device 11 is installed.

For example, the extraction unit 121 acquires three-dimensional acceleration data and angular velocity data in the local coordinate system of the data acquisition device 11. The extraction unit 121 converts the acquired sensor data into the world coordinate system to generate time-series data. For example, the extraction unit 121 generates time-series data of three-dimensional acceleration data or time-series data of three-dimensional angular velocity data converted into the world coordinate system.

For example, the extraction unit 121 generates time-series data such as a spatial acceleration and a spatial angular velocity. Furthermore, the extraction unit 121 integrates the spatial acceleration and the spatial angular velocity, and generates time-series data such as the spatial velocity, the spatial angle (plantar angle), and the sensor height. The extraction unit 121 generates the time-series data at a predetermined timing or time interval set in accordance with a general gait cycle or a gait cycle unique to the user. The timing when the extraction unit 121 generates the time-series data can be arbitrarily set. For example, the extraction unit 121 continues to generate time-series data during a period in which a gait of the user is continued. Furthermore, the extraction unit 121 may be configured to generate time-series data at a specific time.

For example, the extraction unit 121 extracts the time-series data (the gait waveform data) for one gait cycle from the generated time-series data.

The detection unit 123 detects the gait event of the walker walking while wearing footwear on which the data acquisition device 11 is installed from the gait waveform data generated by the extraction unit 121. For example, the detection unit 123 detects the timing of the characteristic maximum value or minimum value in the gait waveform data. For example, the detection unit 123 outputs the detected gait event to a system or a device (not illustrated).

[Walk Event]

Next, the gait event detected by the detection device 12 will be described with reference to the drawings. Hereinafter, an example of verifying the gait of the subject wearing the footwear on which the data acquisition device 11 is installed will be described. In this verification, 51 subjects were set as a population, and the gait of the walker wearing the footwear in which the data acquisition device 11 was installed was measured by the motion capture and the detection device 12. Then, the gait measured by the motion capture was compared with the gait measured by the detection device 12 using the sensor data generated by the data acquisition device 11.

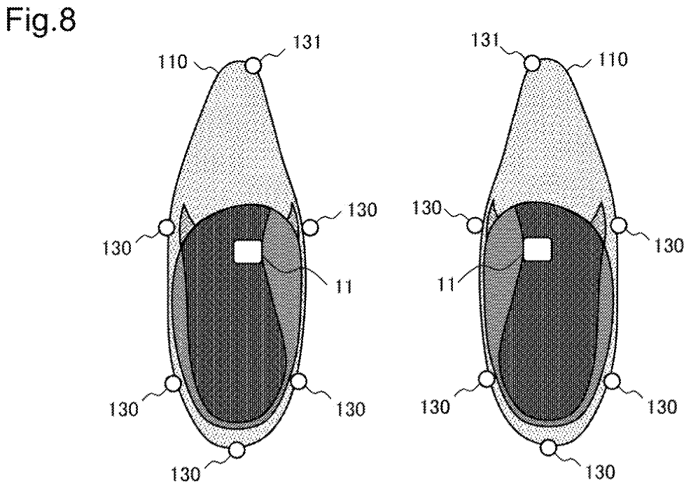

FIG. 8 is a conceptual diagram of the shoe 110 with markers 130 and 131 attached for the motion capture. In this verification, five markers 130 and one marker 131 were attached to each of the shoes 110 of both feet. Five markers 130 were arranged on the side surface around the opening of the shoe. The five markers 130 are for detecting movement of the heel. The center of gravity of the rigid body model that regards the five markers 130 as rigid bodies is detected as the position of the heel. The marker 131 was arranged at the position of the toe of the shoe 110. The marker 131 is detected as the position of the toe. In addition, the data acquisition device 11 was installed at a position corresponding to the back side of the arch of the foot.

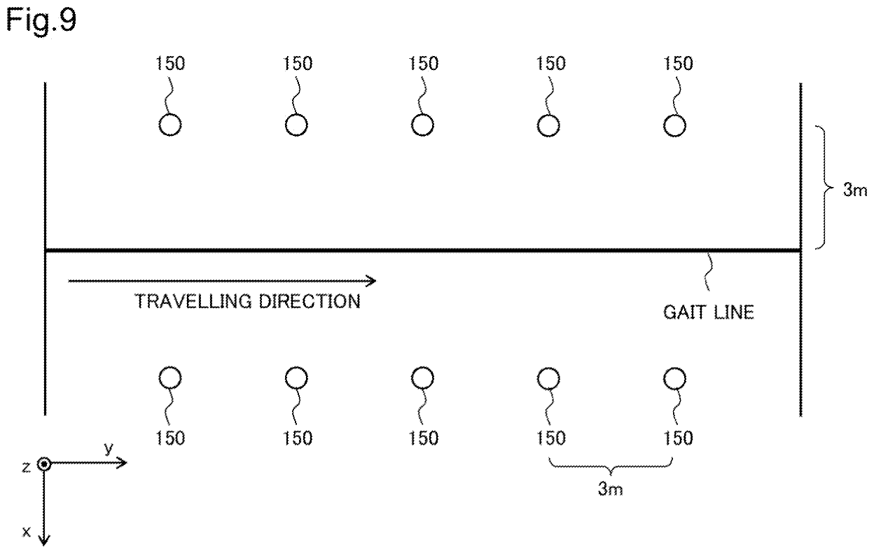

FIG. 9 is a conceptual diagram for illustrating a gait line when the gait of the walker wearing the shoe 110 to which the marker 130 and the marker 131 are attached is verified by the motion capture and positions where the plurality of cameras 150 are disposed. In this verification, 5 cameras 150 (10 cameras in total) were disposed on both sides across the gait line. Each of the plurality of cameras 150 was disposed at an interval of 3 m at a position of 3 m from the gait line. The height of each of the plurality of cameras 150 was fixed at a height of 2 m from a horizontal plane (XY plane). The focal point of each of the plurality of cameras 150 was aligned with the position of the gait line.

The movement of the marker 130 and the marker 131 installed on the shoe 110 of the walker walking along the gait line was analyzed using the moving images captured by the plurality of cameras 150. The movement of the heel was verified by considering the plurality of markers 130 as one rigid body and analyzing the movement of the center of gravity of the markers. The movement of the toe was verified by analyzing the movement of the marker 131. In this verification, the heights (hereinafter, referred to as a height in a Z-direction) of the heel and the toe in the gravity direction were measured.

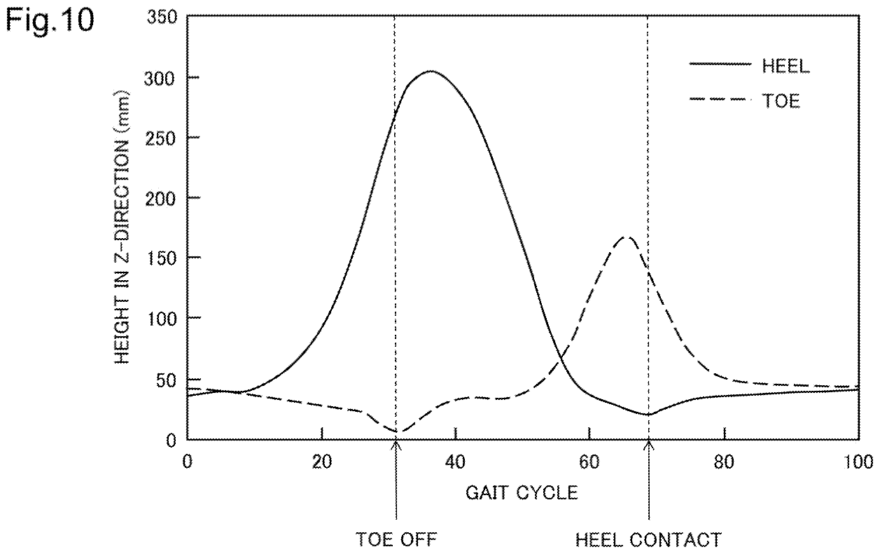

FIG. 10 is a graph illustrating a gait cycle dependency of the heights of the toe and the heel in the Z-direction measured by the motion capture. In FIG. 10, a change in the height of the toe in the Z-direction is indicated by a broken line, and a change in the height of the heel in the Z-direction is indicated by a solid line. The timing when the height of the toe in the Z-direction is minimized is the timing of the too off. The timing when the height of the heel in the Z-direction is minimized is the timing of the heel contact.

<Toe Off>

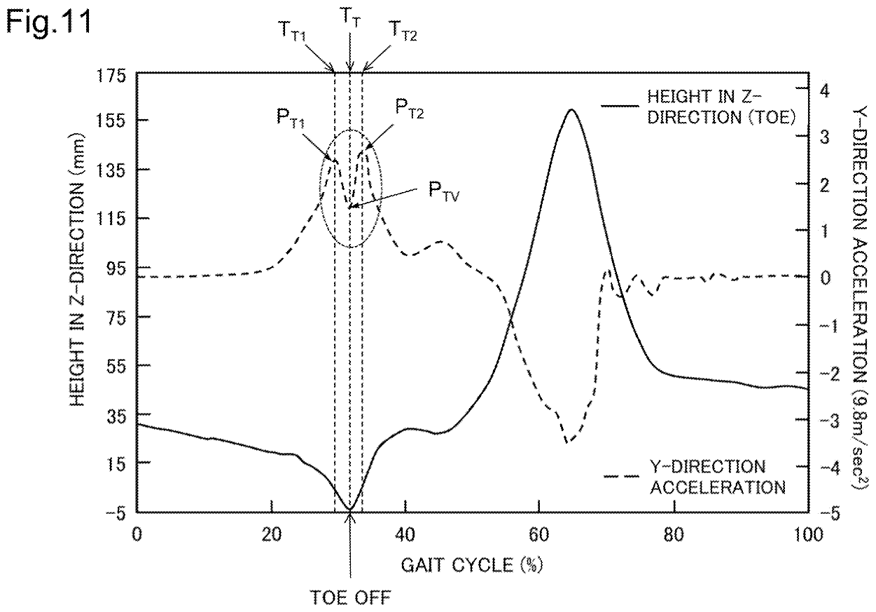

FIG. 11 is a graph in which the height of the toe in the Z-direction measured by the motion capture is associated with the gait waveform data of the Y-direction acceleration measured by the detection device 12 by using the sensor data generated by the data acquisition device 11. A change in the height of the toe in the Z-direction measured by the motion capture is indicated by a solid line. A change in the acceleration in the Y-direction measured by the detection device 12 is indicated by a broken line.

As illustrated in FIG. 11, in the Y-direction acceleration, two maximum peaks (peak P_{T1} and peak P_{T2}) and one minimum peak (peak P_{TV}) were detected at the maximum peak detected around the gait cycle of 20 to 40% (within a range surrounded by a dotted line). The timing of the toe off coincides with the timing T_{T} when the peak P_{TV} is detected between timing T_{T1} when the peak P_{T1} is detected and timing T_{T2} when the peak P_{T2} is detected.

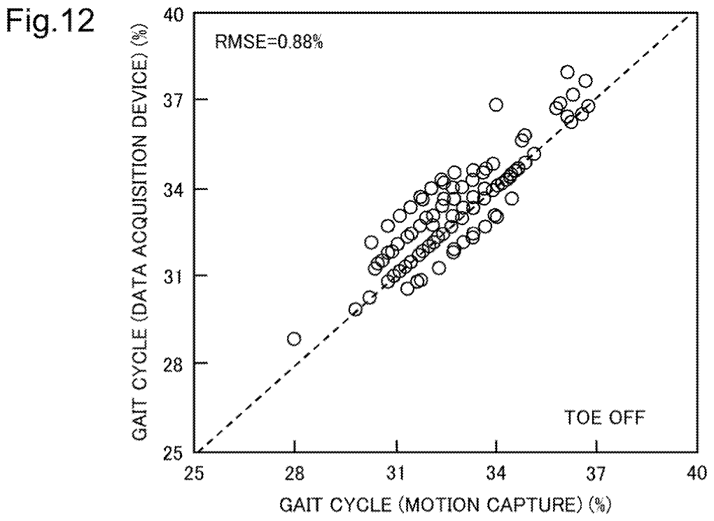

FIG. 12 is a graph for verifying a correlation between the timing of the toe off detected by the motion capture and the timing of the toe off detected by the data acquisition device 11, with 51 subjects as a population. FIG. 12 illustrates a regression line obtained by linearly regressing the timing of the toe off detected by the motion capture and the timing of the toe off detected by the data acquisition device 11. The root mean squared error (RMSE) of the regression line was 0.88%. That is, there is a sufficient correlation between the timing of the toe off detected by the motion capture and the timing of the toe off detected by the data acquisition device 11.

<Heel Separated Place>

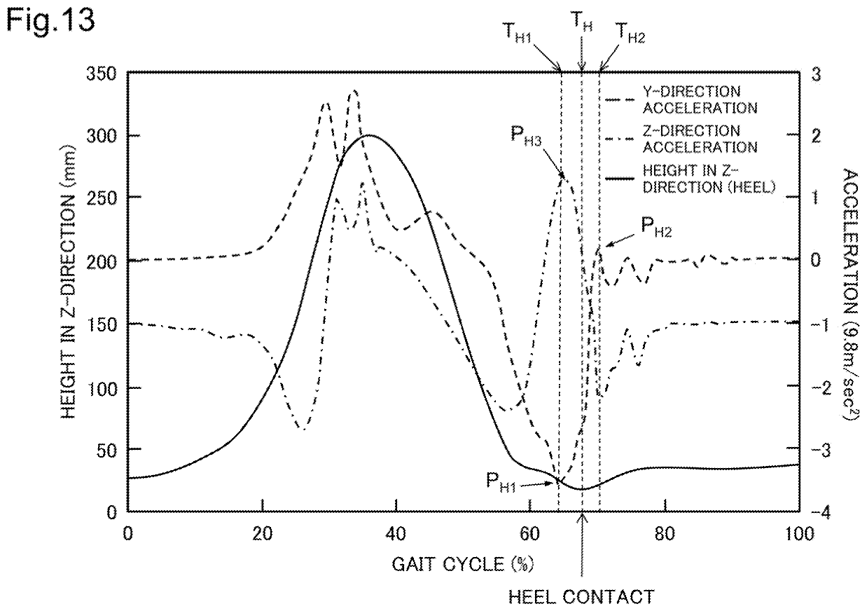

FIG. 13 is a graph in which the height of the heel in the Z-direction measured by the motion capture is associated with the gait waveform data of the Y-direction acceleration and the Z-direction acceleration measured by the detection device 12 by using the sensor data generated by the data acquisition device 11. A change in the height of the heel in the Z-direction measured by the motion capture is indicated by a solid line. A change in the acceleration in the Y-direction measured by the detection device 12 is indicated by a broken line. A change in the acceleration in the Z-direction measured by the detection device 12 is indicated by a one-dot chain line.

As illustrated in FIG. 13, in the Y-direction acceleration, the minimum peak (the peak P_{H1}) was detected around when the gait cycle exceeded 60%. The peak P_{H1} corresponds to the timing of sudden deceleration of the foot at the end of terminal swing period. In addition, in the Y-direction acceleration, the peak P_{H2} at which the gait cycle was maximum around 70% was detected. The peak P_{H2} corresponds to the timing of the heel rocker. The timing of the heel rocker includes a period in which the acceleration in the gravity direction (the Z-direction) is converted into the traveling direction (the Y-direction) by the rotation along the outer periphery of the grounded heel after heel contact. The timing T_{H} of the midpoint between the timing T_{H1} when the peak P_{H1} is detected and the timing T_{H2} when the peak P_{H2} is detected coincides with the timing of heel contact. Instead of the timing T_{H1} when the peak P_{H1} is detected in the Y-direction acceleration, a timing when the maximum peak (peak P_{H3}) when the gait cycle exceeds 60% in the Z-direction acceleration is detected may be used.

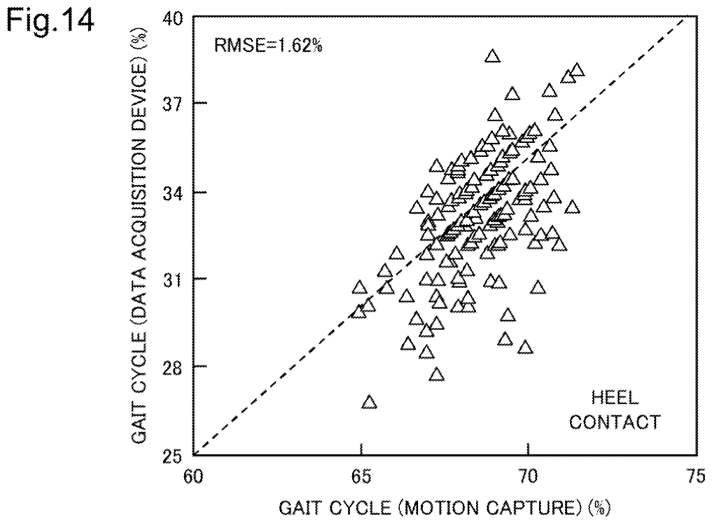

FIG. 14 is a graph for verifying a correlation between the timing of the heel contact detected by the motion capture and the timing of the heel contact detected by the data acquisition device 11, with 51 subjects as a population. FIG. 14 illustrates a regression line obtained by linearly regressing the timing of the heel contact detected by the motion capture and the timing of the heel contact detected by the data acquisition device 11. The root mean square error RMSE of the regression line was 1.62%. That is, there is a sufficient correlation between the timing of the heel contact detected by the motion capture and the timing of the heel contact detected by the data acquisition device 11.

The detection unit 123 specifies a timing of the gait event different from the toe off or the heel contact with reference to a timing of at least one of the toe off and the heel contact. As in FIG. 5, the gait event is associated with the gait cycle. The periods of the stance phase and the swing phase can be specified with reference to the heel contact (a) and the toe off (e). In addition, with reference to the heel contact (a) and the toe off (e), the timing of the gait event such as the opposite foot toe off (b), the heel lift (c), the opposite foot heel contact (d), and the foot crossing (f) can be specified. In addition, with reference to the heel contact (a) and the toe off (e), the timing of the gait event different from the opposite foot toe off (b), the heel lift (c), the opposite foot heel contact (d), the foot crossing (f), and the like can be specified. If the timing of the gait event can be specified, the movement of the foot, the angle of the foot, the force applied to the foot, and the like at each timing can be verified. The timing of the gait event detected by the detection unit 123 may be output to another system, a display device, or the like (not illustrated). The timing of the gait event detected by the detection unit 123 can be used for various purposes of measuring the gait.

(Operation)

Next, an operation of the detection system 1 of the present example embodiment will be described with reference to the drawings. Hereinafter, the extraction unit 121 and the detection unit 123 of the detection system 1 are mainly operated. The subject of the operation described below may be the detection system 1.

[Extraction Unit]

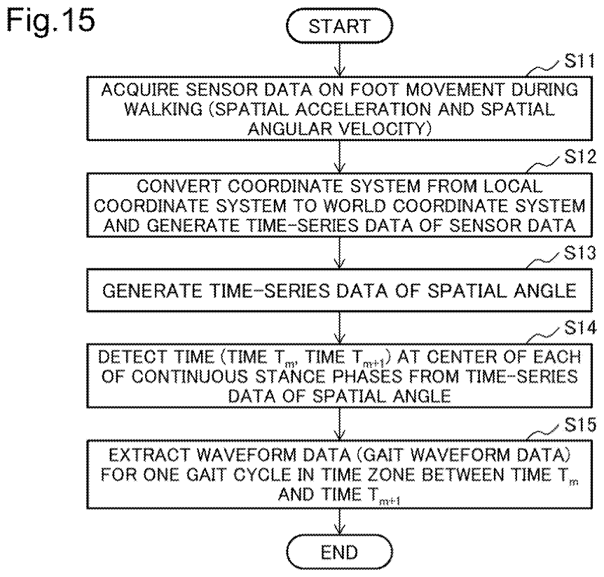

First, the operation of the extraction unit 121 of the detection system 1 will be described with reference to the drawings. FIG. 15 is a flowchart for illustrating an example of the operation of the extraction unit 121.

In FIG. 15, first, the extraction unit 121 acquires, from the data acquisition device 11, sensor data regarding the movement of the foot of the walker walking while wearing the footwear on which the data acquisition device 11 is installed (step S11). The extraction unit 121 acquires sensor data in a local coordinate system of the data acquisition device 11. For example, the extraction unit 121 acquires a three-dimensional spatial acceleration and a three-dimensional spatial angular velocity from the data acquisition device 11 as sensor data regarding the movement of the foot.

Next, the extraction unit 121 converts the coordinate system of the acquired sensor data from the local coordinate system to the world coordinate system, and generates time-series data of the sensor data (step S12).

Next, the extraction unit 121 calculates the spatial angle using at least one of the spatial acceleration and the spatial angular velocity and generates the time-series data of the spatial angle (step S13). The extraction unit 121 generates time-series data of the spatial velocity and the spatial trajectory as necessary. Step S13 may be performed before step S12.

Next, the extraction unit 121 detects the time (time tₘ, time tₘ₊₁) at the center of each of the continuous stance phases from the time-series data of the spatial angle (step S14).

Next, the extraction unit 121 extracts the waveform data (the gait waveform data) for one gait cycle in the time period between the time tₘ and the time tₘ₊₁ from the time-series data of the spatial acceleration and the spatial angular velocity of the detection target of the gait event (step S15).

[Detection Unit]

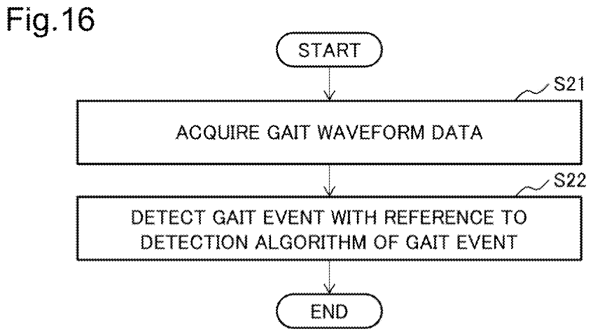

Next, the operation of the detection unit 123 of the detection system 1 will be described with reference to the drawings. FIG. 16 is a flowchart for illustrating an example of the operation of the detection unit 123.

In FIG. 16, first, the detection unit 123 acquires the gait waveform data generated by the extraction unit 121 (step S21).

Next, the detection unit 123 detects the gait event from the gait waveform data with reference to a detection algorithm of the gait event (step S22). For example, the detection unit 123 refers to an algorithm for detecting a gait event such as toe off or heel contact stored in a database (not illustrated). For example, the detection algorithm includes an algorithm for detecting the start timing of the swing phase and an algorithm for detecting the start timing of the stance phase.

<Swing Phase>

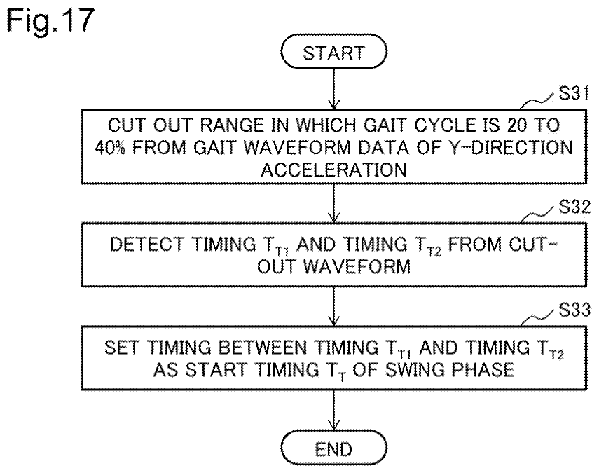

Next, an algorithm for detecting the start of the swing phase will be described with reference to the drawings. FIG. 17 is a flowchart for illustrating an example of an algorithm for detecting the toe off as the start timing of the swing phase. Hereinafter, the detection unit 123 will be described as a main body of the operation, but the detection device 12 may be a subject of the operation.

In FIG. 17, first, the detection unit 123 cuts out a range in which the gait cycle is 20 to 40% from the gait waveform data of the Y-direction acceleration (step S31).

Next, the detection unit 123 detects the timing T_{T1} and the timing T_{T2} from the cut-out waveforms (step S32).

Then, the detection unit 123 sets the timing of the midpoint between the timing T_{T1} and the timing T_{T2} as the start timing T_{T} of the swing phase (step S33).

<Stance Phase>

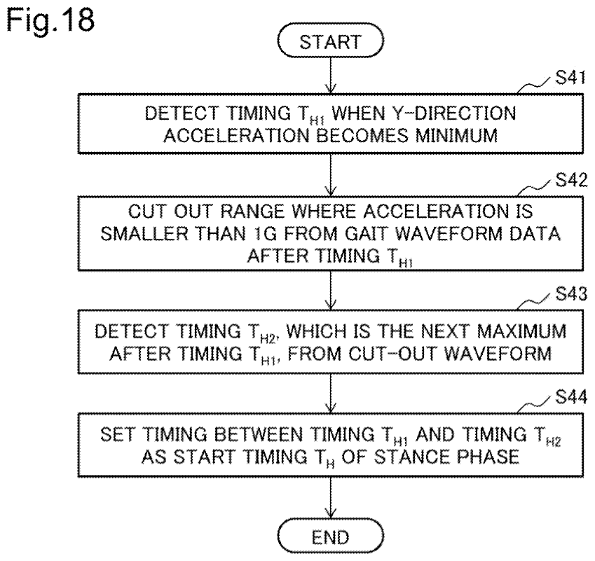

Next, an algorithm for detecting the start of the stance phase will be described with reference to the drawings. FIG. 18 is a flowchart for illustrating an example of an algorithm for detecting the heel contact as the start timing of the stance phase. Hereinafter, the detection unit 123 will be described as a main body of the operation, but the detection device 12 may be a subject of the operation.

In FIG. 18, first, the detection unit 123 detects the timing T_{H1} when the Y-direction acceleration becomes minimum from the gait waveform data of the Y-direction acceleration (step S41).

Next, the detection unit 123 cuts out a range in which the value of the Y-direction acceleration becomes smaller than 1 G after the timing T_{H1} from the gait waveform data of the Y-direction acceleration (step S42).

Next, the detection unit 123 detects the timing T_{H1} and the timing T_{H2} from the cut-out waveforms (step S43).

Then, the detection unit 123 sets the timing of the midpoint between the timing T_{H1} and the timing T_{H2} as the start timing T_{H} of the stance phase (step S44).

The operation of the detection system 1 has been described above. The processing along the flowcharts of FIGS. 15 to 18 is an example, and does not limit the operation of the detection system 1.

As described above, the detection device includes the extraction unit and the detection unit. The extraction unit extracts waveform data based on a gait of a walker using sensor data acquired from a sensor installed on a foot of the walker. The detection unit detects a gait event from the waveform data extracted by the extraction unit.

According to the present example embodiment, it is possible to detect a gait event in a gait of a walker based on data acquired by a data acquisition device (a sensor) installed on a foot of the walker. For example, the detection device detects the timing of a gait event such as a toe off or heel contact in time-series data of sensor data generated based on a gait of a walker.

In one aspect of the present example embodiment, the extraction unit acquires at least one of the spatial acceleration and the spatial angular velocity as sensor data. The extraction unit extracts gait waveform data, which is waveform data for one gait cycle of the walker, from time-series data of sensor data of at least one of the spatial acceleration and the spatial angular velocity.

In one aspect of the present example embodiment, the detection unit detects the gait event based on the peak of the gait waveform data. According to the present aspect, by detecting the gait event based on the peak of the gait waveform data, the reference of the measurement of the gait of the walker becomes clear, and thus, it is possible to measure the gait more accurately.

In one aspect of the present example embodiment, the extraction unit acquires an acceleration of a walker in a traveling direction as sensor data and generates gait waveform data from time-series data of the acceleration in the traveling direction. The detection unit detects a timing when a valley is detected between two peaks included in the maximum peak of the gait waveform data of the acceleration in the traveling direction as the timing of the too off. According to the present aspect, the timing of the toe off can be detected as the gait event based on the acceleration in the traveling direction.

In one aspect of the present example embodiment, the extraction unit acquires an acceleration of a walker in a traveling direction as sensor data and generates gait waveform data from time-series data of the acceleration in the traveling direction. The detection unit detects a timing of a midpoint between a timing when the minimum peak of the gait waveform data of the acceleration in the traveling direction is detected and a timing when the maximum peak appearing after the minimum peak is detected as a timing of heel contact. According to the present aspect, the timing of the heel contact can be detected as the gait event based on the acceleration in the traveling direction.

In one aspect of the present example embodiment, the extraction unit acquires the acceleration in the traveling direction and the acceleration in the gravity direction of the walker as sensor data, and generates gait waveform data from the time-series data of the acceleration in the traveling direction and the time-series data of the acceleration in the gravity direction. The detection unit detects the timing of the midpoint between the timing when the minimum peak of the acceleration in the gravity direction is detected and the timing when the maximum peak appearing after the minimum peak of the gait waveform data of the acceleration in the traveling direction is detected as the timing of heel contact. According to the present aspect, the timing of the heel contact can be detected as the gait event based on the acceleration in the traveling direction and the acceleration in the gravity direction.

In an aspect of the present example embodiment, the detection unit specifies a timing of the gait event different from the toe off or the heel contact with reference to a timing of at least one of the toe off or the heel contact. According to the present aspect, the timing of other gait events can be accurately detected with reference to the timing of the toe off and the heel contact.

A detection system according to an aspect of the present example embodiment includes a data acquisition device and a detection device. The data acquisition device measures a spatial acceleration and a spatial angular velocity. The data acquisition device generates sensor data based on the measured spatial acceleration and spatial angular velocity. The data acquisition device transmits the generated sensor data to the detection device. The detection device extracts waveform data based on a gait of a walker using sensor data acquired from a sensor installed on a foot of the walker. The detection device detects the gait event from the extracted waveform data. According to the present aspect, the timing of the gait event can be detected using the spatial acceleration and the spatial angular velocity measured by the data acquisition device.

### Second Example Embodiment

Next, a detection device according to a second example embodiment will be described with reference to the drawings. The detection device of the present example embodiment has a simplified configuration of the detection device 12 of the first example embodiment.

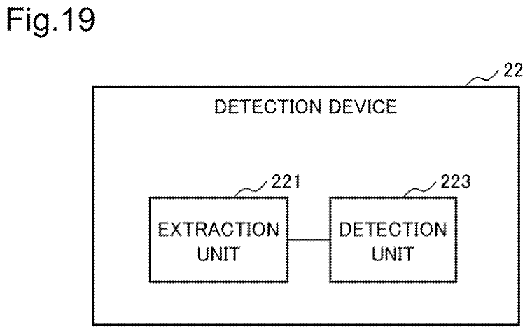

FIG. 19 is a block diagram illustrating an example of a configuration of a detection device 22 in the present example embodiment. The detection device 22 includes an extraction unit 221 and a detection unit 223. The extraction unit 221 extracts waveform data based on a gait of a walker using sensor data acquired from a sensor installed on a foot of the walker. The detection unit 223 detects a gait event from the waveform data extracted by the extraction unit 221.

According to the detection device of the present example embodiment, it is possible to detect a gait event in a gait of a walker based on data acquired by a sensor installed on a foot of the walker. For example, the detection device of the present example embodiment detects the timing of a gait event such as a toe off or heel contact in time-series data of sensor data generated based on a gait of a walker.

(Hardware)

Here, a hardware configuration for executing the processing of the detection device according to the example embodiment will be described using an information processing apparatus 90 of FIG. 19 as an example. The information processing apparatus 90 in FIG. 19 is a configuration example for executing processing of the detection device of each example embodiment, and does not limit the scope of the present invention.

As illustrated in FIG. 19, the information processing apparatus 90 includes a processor 91, a main storage device 92, an auxiliary storage device 93, an input/output interface 95, and a communication interface 96. In FIG. 19, the interface is abbreviated as an interface (I/F). The processor 91, the main storage device 92, the auxiliary storage device 93, the input/output interface 95, and the communication interface 96 are data-communicably connected to each other via a bus 99. In addition, the processor 91, the main storage device 92, the auxiliary storage device 93, and the input/output interface 95 are connected to a network such as the Internet or an intranet via the communication interface 96.

The processor 91 develops the program stored in the auxiliary storage device 93 or the like in the main storage device 92 and executes the developed program. In the present example embodiment, a software program installed in the information processing apparatus 90 may be used. The processor 91 executes processing by the detection device according to the present example embodiment.

The main storage device 92 has an area in which a program is developed. The main storage device 92 may be a volatile memory such as a dynamic random access memory (DRAM). In addition, a nonvolatile memory such as a Magnetoresistive Random Access Memory (MRAM) may be configured and added as the main storage device 92.

The auxiliary storage device 93 stores various data. The auxiliary storage device 93 includes a local disk such as a hard disk or a flash memory. Various data may be stored in the main storage device 92, and the auxiliary storage device 93 may be omitted.

The input/output interface 95 is an interface for connecting the information processing apparatus 90 and a peripheral device. The communication interface 96 is an interface for connecting to an external system or device through a network such as the Internet or an intranet on the basis of a standard or a specification. The input/output interface 95 and the communication interface 96 may be shared as an interface connected to an external device.

An input device such as a keyboard, a mouse, or a touch panel may be connected to the information processing apparatus 90 as necessary. These input devices are used to input information and settings. When the touch panel is used as the input device, the display screen of the display device may also serve as the interface of the input device. Data communication between the processor 91 and the input device may be mediated by the input/output interface 95.

Furthermore, the information processing apparatus 90 may be provided with a display device for displaying information. In a case where a display device is provided, the information processing apparatus 90 preferably includes a display control device (not illustrated) for controlling display of the display device. The display device may be connected to the information processing apparatus 90 via the input/output interface 95.

The above is an example of a hardware configuration for enabling the detection device according to each example embodiment of the present invention. The hardware configuration of FIG. 19 is an example of a hardware configuration for executing arithmetic processing of the detection device according to each example embodiment, and does not limit the scope of the present invention. In addition, a program for causing a computer to execute processing related to the detection device according to each example embodiment is also included in the scope of the present invention.

Further, a non-transitory recording medium (also referred to as a program recording medium) in which the program according to each example embodiment is recorded is also included in the scope of the present invention. For example, the recording medium can be achieved by an optical recording medium such as a Compact Disc (CD) or a Digital Versatile Disc (DVD). Furthermore, the recording medium may be achieved by a semiconductor recording medium such as a Universal Serial Bus (USB) memory or a Secure Digital (SD) card, a magnetic recording medium such as a flexible disk, or another recording medium.

The components of the detection device of each example embodiment can be arbitrarily combined. In addition, the components of the detection device of each example embodiment may be achieved by software or may be achieved by a circuit.

While the invention has been particularly shown and described with reference to exemplary embodiments thereof, the invention is not limited to these embodiments. It will be understood by those of ordinary skill in the art that various changes in form and details may be made therein without departing from the spirit and scope of the present invention as defined by the claims.

### REFERENCE SIGNS LIST

1 detection system
11 data acquisition device
12, 22 detection device
111 acceleration sensor
112 angular velocity sensor
113 signal processing unit
115 data transmission unit
121, 221 extraction unit
123, 223 detection unit

What is claimed is:

1. A detection device comprising:
at least one memory storing instructions; and
at least one processor connected to the at least one memory and configured to execute the instructions to:
extract waveform data based on a gait of a walker using sensor data acquired from a sensor installed on a foot of the walker; and
detect a gait event from the waveform data extracted by the extraction means.

2. The detection device according to claim 1, wherein
the at least one processor is configured to execute the instructions to
acquire at least one of a spatial acceleration and a spatial angular velocity as the sensor data and
extract gait waveform data that is the waveform data for one gait cycle of the walker from time-series data of the sensor data of at least one of the spatial acceleration and the spatial angular velocity.

3. The detection device according to claim 2, wherein
the at least one processor is configured to execute the instructions to detect the gait event based on a peak of the gait waveform data.

4. The detection device according to claim 2, wherein
the at least one processor is configured to execute the instructions to
acquire an acceleration of the walker in a traveling direction as the sensor data and
generate the gait waveform data from time-series data of the acceleration in the traveling direction, and
detect a timing when a valley is detected between two peaks included in the maximum peak of the gait waveform data of the acceleration in the traveling direction as a timing of too off.

5. The detection device according to claim 4, wherein
the at least one processor is configured to execute the instructions to
acquire an acceleration of the walker in the traveling direction as the sensor data and
generate the gait waveform data from time-series data of the acceleration in the traveling direction, and
detect a timing of a midpoint between a timing when the minimum peak of the gait waveform data of the acceleration in the traveling direction is detected and a timing when the maximum peak appearing after the minimum peak is detected as a timing of heel contact.

6. The detection device according to claim 4, wherein
the at least one processor is configured to execute the instructions to
acquire an acceleration in a traveling direction and an acceleration in a gravity direction of the walker as the sensor data and
generate the gait waveform data from the time-series data of the acceleration in the traveling direction and the time-series data of the acceleration in the gravity direction, and
detect a timing of a midpoint between a timing when the minimum peak of the acceleration in the gravity direction is detected and a timing when the maximum peak appearing after the minimum peak of the gait waveform data of the acceleration in the traveling direction is detected as a timing of heel contact.

7. The detection device according to claim 5, wherein
the at least one processor is configured to execute the instructions to specify a timing of the gait event different from the toe off or the heel contact with reference to a timing of at least one of the toe off or the heel contact.

8. A detection system comprising:
the detection device according to claim 1; and
a data acquisition device that measures a spatial acceleration and a spatial angular velocity, generates the sensor data based on the measured spatial acceleration and spatial angular velocity, and transmits the generated sensor data to the detection device.

9. A detection method performed by a computer, the method comprising:
extracting waveform data based on a gait of a walker using sensor data acquired from a sensor installed on a foot of the walker; and
detecting a gait event from the extracted waveform data.

10. A non-transitory program recording medium storing a program for causing a computer to execute:
a process for extracting waveform data based on a gait of a walker using sensor data acquired from a sensor installed on a foot of the walker; and
a process for detecting a gait event from the extracted waveform data.
